(19) United States
(12) Patent Application Publication
(10) Pub. No.: US 2009/0131924 A1
(43) Pub. Date: May 21, 2009

(54) Endovascular Thermal Treatment Device with Carrier Wire and Method

(76) Inventors: Ralph A. Meyer, Argyle, NY (US); William M. Appling, Granville, NY (US)

Correspondence Address: AFS / ANGIODYNAMICS, 666 THIRD AVENUE, FLOOR 10, NEW YORK, NY, 10017, US

(21) Appl. No.: 12/271,449

(22) Filed: Nov. 14, 2008

Related U.S. Application Data
(60) Provisional application No. 60/988,625, filed on Nov. 16, 2007.

(51) Int. Cl. A61B 18/22; A61B 18/18

(52) U.S. Cl. 606/15; 606/33

(57) ABSTRACT

An endovenous thermal treatment device in which a thermal energy delivery device can be inserted into a blood vessel without the use of a treatment sheath is provided. The treatment device includes a carrier wire having a flexible distal section and a longitudinal energy delivery device having a distal energy emitting section. A coupler couples the carrier wire and the energy delivery device so that they are inserted together into a blood vessel.

### CROSS REFERENCE TO RELATED APPLICATIONS

This application claims the benefit of priority under 35 U.S.C. Section 119(e) to U.S. Provisional Application Ser. No. 60/988,625, filed Nov. 16, 2007, entitled “Endovascular Thermal Treatment Device With Carrier Wire And Method”, which is fully incorporated by reference herein.

### FIELD OF THE INVENTION

The present invention relates to a medical apparatus and method for treatment of blood vessels. More particularly, the present invention relates to an endovascular apparatus and method for minimally invasive treatment of venous reflux disease.

### BACKGROUND OF THE INVENTION

Veins can be broadly divided into three categories: the deep veins, which are the primary conduit for blood return to the heart; the superficial veins, which parallel the deep veins and function as a channel for blood passing from superficial structures to the deep system; and topical or cutaneous veins, which carry blood from the end organs (e.g., skin) to the superficial system. Veins have thin walls and contain one-way valves that control blood flow. Normally, the valves open to allow blood to flow into the deep veins and close to prevent back-flow into the superficial veins. When the valves are malfunctioning or only partially functioning, however, they no longer prevent the back-flow of blood into the superficial veins. This condition is called reflux. As a result of reflux, venous pressure builds within the superficial system. This pressure is transmitted to topical veins, which, because the veins are thin walled and not able to withstand the increased pressure, become dilated, tortuous or engorged.

In particular, venous reflux in the lower extremities is one of the most common medical conditions of the adult population. It is estimated that venous reflux disease affects approximately 25% of adult females and 10% of adult males. Symptoms of reflux include varicose veins and other cosmetic deformities, as well as aching and swelling of the legs. Varicose veins are common in the superficial veins of the legs, which are subject to high pressure when standing. Aside from being cosmetically undesirable, varicose veins are often painful, especially when standing or walking. If left untreated, venous reflux may cause severe medical complications such as bleeding, phlebitis, ulcerations, thrombi and lipodermatosclerosis (LDS).

When veins become enlarged, the leaflets of the valves no longer meet properly. Blood collects in the superficial veins, which become even more enlarged. Since most of the blood in the legs is returned by the deep veins, and the superficial veins only return about 10%, they can be removed or closed down without serious harm. Endovascular thermal therapy is a minimally invasive treatment involving the delivery of thermal energy generated by laser, or radio or microwave frequencies, to cause vessel occlusion or ablation. Thermal energy is delivered to the vein wall or blood (depending on the device and method of treatment) using an energy source that is placed within the vein and withdrawn while the energy is emitted. The device and method of treatment can vary significantly depending on the type of energy used. For example, devices that employ laser energy involve inserting a fiber optic line into the vein to deliver laser energy to the blood within the vein to heat the blood and, in turn, heat the walls of the vein. Contact between the emitting face of the fiber and the vein wall is typically avoided in order to prevent perforating the vein and the pain and bruising associated with such perforations. In RF devices, on the other hand, a device with electrodes is inserted into the vein. In order for such devices to work, and in contrast to laser devices, the electrodes must be placed into contact with the vein wall and maintained in contact throughout the delivery of the RF energy. Thus, RF devices are significantly different than laser devices, and the associated methods involve different steps.

Current endovenous treatment using either laser or RF energy requires numerous steps and medical components. A typical laser procedure involves the following steps as shown in FIG. 5A. First, a target vein is accessed using a standard Seldinger technique. In step 100, a vein is accessed using a small gauge needle. A 0.018″ guidewire is then inserted into the lumen of the needle and advanced into the vein (step 102). Once access is gained, the needle is removed and a relatively short micropuncture sheath/dilator set (e.g., 6.15″ dilator length with 4.125″ sheath length) is advanced over the guidewire and into the vein (step 104). Typically, the sheath/dilator set is a 5 F size in order to allow insertion of a 0.035″ procedure guidewire. The dilator and the 0.018″ guidewire are removed (step 106) to leave the micropuncture sheath in place and the larger 0.035″ guidewire is inserted into the vein through the micropuncture sheath (step 108). The micropuncture sheath is then removed, leaving just the 0.035″ guidewire in place (step 110). A longer, larger treatment sheath with dilator, typically a 6 F or larger size sheath, is then threaded over the 0.035″ guidewire into the vein (step 112). The treatment dilator is removed (step 114) and then the 0.035″ guidewire is removed (step 116) to leave the treatment sheath in place. Through the treatment sheath, an energy delivery device such as an optical fiber is inserted and advanced until the fiber face at its distal end is flush with the distal end of the treatment sheath (step 118). The treatment sheath is then retracted so as to expose the distal end of the fiber (step 120). Once both the fiber and sheath are positioned, the user administers tumescent anesthesia along the vein to be treated (step 122). If necessary, the fiber tip position may be adjusted after tumescent anesthesia delivery. The last step of the procedure is to pull back the fiber and sheath together through the vein while energy is emitted from the emitting face at the distal tip of the fiber (step 124).

A typical procedure takes between 45 minutes to 90 minutes, depending on the patient's anatomy, length of the treatment vein and other procedural factors. Of the total procedure time, only between about 3 to 7 minutes is devoted to the actual application of laser energy within the vein. The majority of the procedure time is devoted to accessing the vein, placing the fiber, and administering tumescent anesthesia.

Therefore, it would be desirable to provide an endovascular treatment device and method which reduces the number of procedural steps required to complete the treatment. The reduction in the required number of procedural steps provides potentially many advantages including reduced overall procedure time, thereby reducing physician costs, reduced complication rates and reduced medical component costs.

### SUMMARY OF THE DISCLOSURE

An endovenous thermal treatment device includes a carrier wire having a flexible distal section and a longitudinal energy delivery device having a distal energy emitting section. A coupler couples the carrier wire and the longitudinal energy delivery device together so that they are inserted together through a blood vessel. The treatment device eliminates the need for a treatment sheath, accessory procedural components and the procedural steps associated with these components.

Other advantages of the apparatus and method of the present invention will become more readily apparent in view of the following detailed description of the invention and accompanying drawings.

### BRIEF DESCRIPTION OF THE DRAWINGS

FIG. 1 is a plan view of an energy delivery device with a removable carrier wire device of the present invention.

FIG. 2A is a plan view of the energy delivery device of the present invention.

FIG. 2B is a partial cross-sectional view of the energy delivery device of FIG. 2A.

FIG. 3A is a plan view of the removable carrier wire device of the present invention.

FIG. 3B is a partial cross-sectional view of the carrier wire device of FIG. 3A.

FIG. 4A is a plan view and cross-sectional view of a removable coupler that couples the carrier wire device and the energy delivery device together.

FIG. 4B is a cross-sectional view of a handle attached to the optical fiber.

FIG. 5A is a flowchart illustrating a prior art endovenous treatment procedure.

FIG. 5B is a flowchart illustrating an improved endovenous treatment procedure in accordance with the present invention.

### DETAILED DESCRIPTION OF THE INVENTION

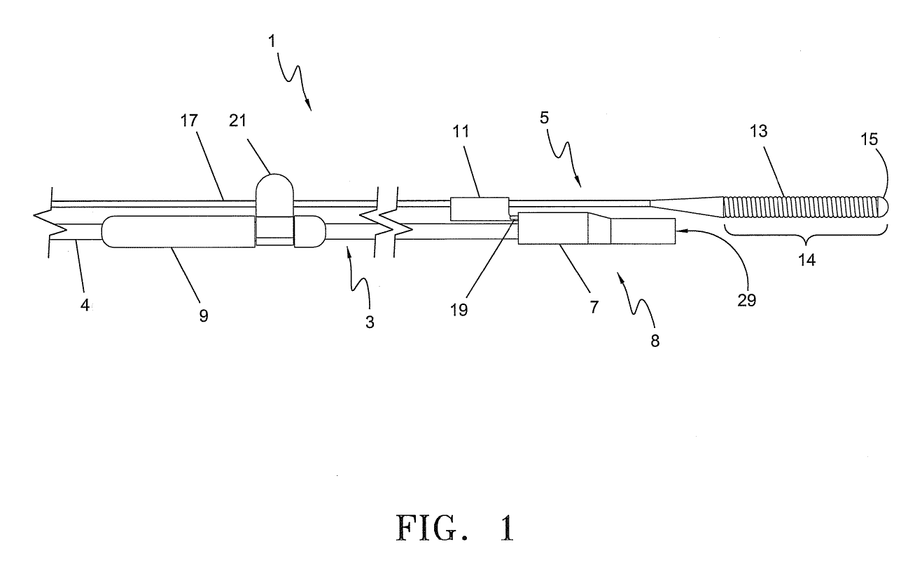

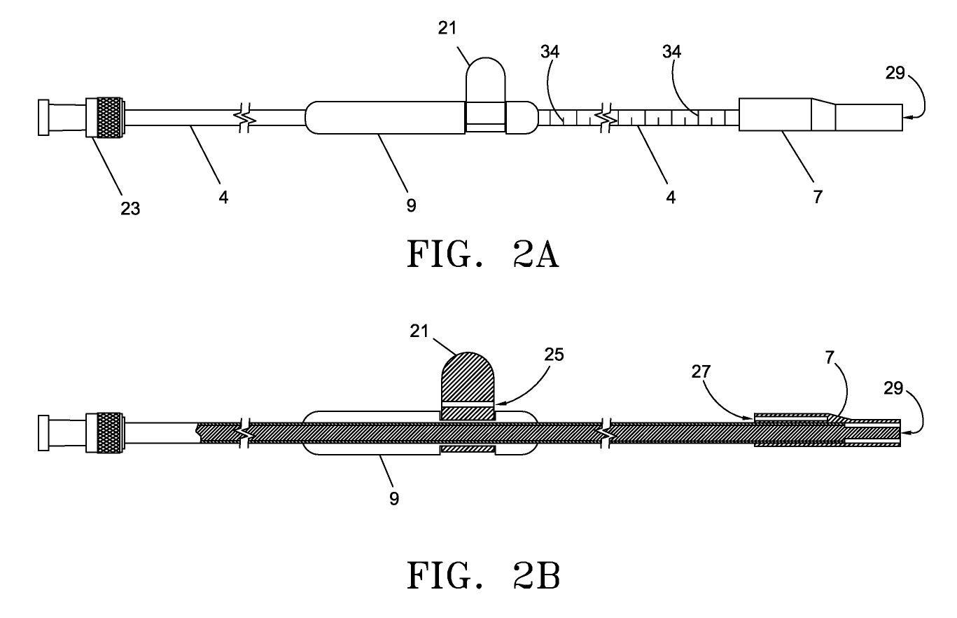

An endovascular treatment device 1 according to the present invention is illustrated in FIG. 1. Device 1 includes an energy delivery device such as an optical fiber device 3 releasably coupled to a carrier wire device 5. An optical fiber 4, as known in the art, has a core, cladding layer surrounding the core and a protective jacket layer surrounding the cladding layer. Attached to the distal portion of the optical fiber device 3 is an energy emitting section 8 including a protective sleeve 7 and an energy emitting face 29 such as described in U.S. Provisional Patent Application Ser. No. 60/913,767, filed Apr. 24, 2007 ('767 Application), which is incorporated herein by reference. The optical fiber device 3 includes a handle 9 which is coaxially mounted upon a proximal section of the optical fiber 4. The handle 9 is coupled to a removable proximal coupler 21 through which a carrier wire shaft 17 is mounted. The optical fiber 4 terminates in a SMA connector 23 (shown in FIG. 2A) for attachment to a laser generator (not shown) during treatment. The carrier wire device 5 is comprised of a guidewire 17 that has been modified to include the removable proximal coupler 21 and a wire-to-sleeve connector 11 having an extension element 19. The carrier wire device 5 includes a flexible distal section 14 that consists of a compression spring or coil 13 and a carrier wire distal tip 15. The distal tip 15 may be more ultrasonically visible than the optical fiber 4. The wire-to-sleeve connector 11 and the coupler 21 releasably connect the optical fiber device 3 to the carrier wire device 5 at the energy emitting section 8 and proximal section of the optical fiber device 3, respectively, which will be more fully described below.

FIG. 2A illustrates a plan view of the optical fiber device 3 component of the endovascular device 1. FIG. 2B depicts a cross-section of FIG. 2A. The optical fiber device 3 is comprised of an optical fiber 4 that extends distally from SMA connector 23 through handle 9, to protective sleeve 7. The optical fiber 3 terminates in an energy emitting front face 29 through which laser energy is emitted as described in the '767 Application. The handle 9 is preferably of a hard polymer having a lumen through which the optical fiber 4 extends. The handle 9 is permanently mounted on the fiber 4 using an adhesive or other attachment technique known in the art. Removably mounted on handle 9 is a removable coupler 21, which functions to connect the carrier wire 17 to the optical fiber device 3 during insertion and advancement of device 1 through a vessel. The coupler 21 has a carrier wire lumen 25 through which the carrier wire shaft 17 is mounted in a fixed position. Alternatively, the carrier wire shaft 17 may be slidable within the lumen 25 such that the wire shaft can be removed from the patient body without detaching the coupler 21 from the handle 9. As will be described in greater detail below, the coupler 21 may be removed from handle 9 to detach the carrier wire 17 from optical fiber 3 thereby allowing removal of the carrier wire device 5 from the vein prior to thermally treating the vessel with laser energy. The optical fiber device 3 includes graduated markings 34 positioned along the fiber shaft 4 from the handle 9 to the sleeve 7.

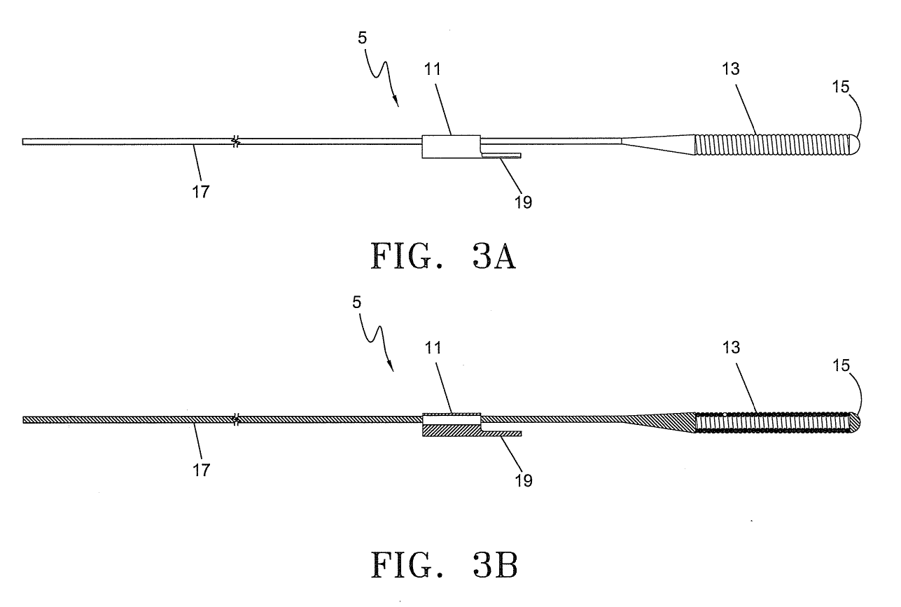

The protective sleeve 7 of the energy emitting section 8 is circumferentially arranged around the distal segment of optical fiber 3. In the embodiment shown, protective sleeve 7 is comprised of metal such as stainless steel so as to provide enhanced ultrasonic visibility of the fiber tip within the energy emitting section 8 and to protect the energy emitting face 29 of the fiber 4 from contact with the wall of the blood vessel. Sleeve 7 also functions to protect the fragile fiber tip from damage during treatment. Shown in FIG. 2B is a longitudinal key slot 27 formed within the wall of protective sleeve 7 and opening in a proximal direction. When carrier wire device 5 is assembled with optical fiber 3, the wire-to-sleeve connector extension element 19 (shown in FIGS. 3A and 3B) is inserted into key slot 27 to removably connect the carrier wire device 5 to the fiber 3.

FIGS. 3A and 3B illustrate a carrier wire device 5. The carrier wire device 5 is comprised of a carrier wire shaft 17, a wire-to-sleeve connector 11 fixedly attached to shaft 17, compression spring or coil 13, and carrier wire distal tip 15. The wire-to-sleeve connector 11 includes an extension element 19 that extends in a longitudinal and distal direction. The wire-to-sleeve connector 11 and key slot 27 comprise a distal coupler that couples the carrier wire device 5 to the optical fiber device 3. Shaft 17 is preferably a mandrel wire of stainless steel with an outer diameter of 0.018″, although other sizes are of course possible.

Alternatively, the treatment device 1 may include multiple wire-to-sleeve connectors and corresponding extension elements. For example, the treatment device 1 may include two wire-to-sleeve connectors 11 longitudinally spaced from each other and both fixedly attached to the optical fiber shaft 17, and two corresponding longitudinally spaced key slots 27 on the sleeve 7 to receive the two extension elements. Still in another alternative, the treatment device 1 may include a wire-to-sleeve connector 11 with two circumferentially spaced extension elements, and two corresponding circumferentially spaced key slots on the sleeve 7.

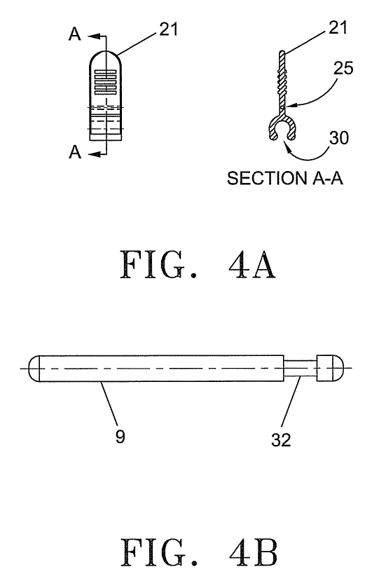

FIG. 4A is a plan view and cross-sectional view of the removable coupler 21 that couples the carrier wire device 5 and the energy delivery device 3 together. The coupler 21 has a cylindrical recess 30 which locks with a reduced diameter cylindrical portion 32 (shown in FIG. 4B) of the handle 9 in an interference fit manner. The width of the coupler 21 is about the same as the length of the reduced diameter cylindrical portion 32 such that the carrier wire device 5 stays tightly locked with the optical fiber device 3 in a longitudinal direction to prevent any longitudinal movement of one device from the other device.

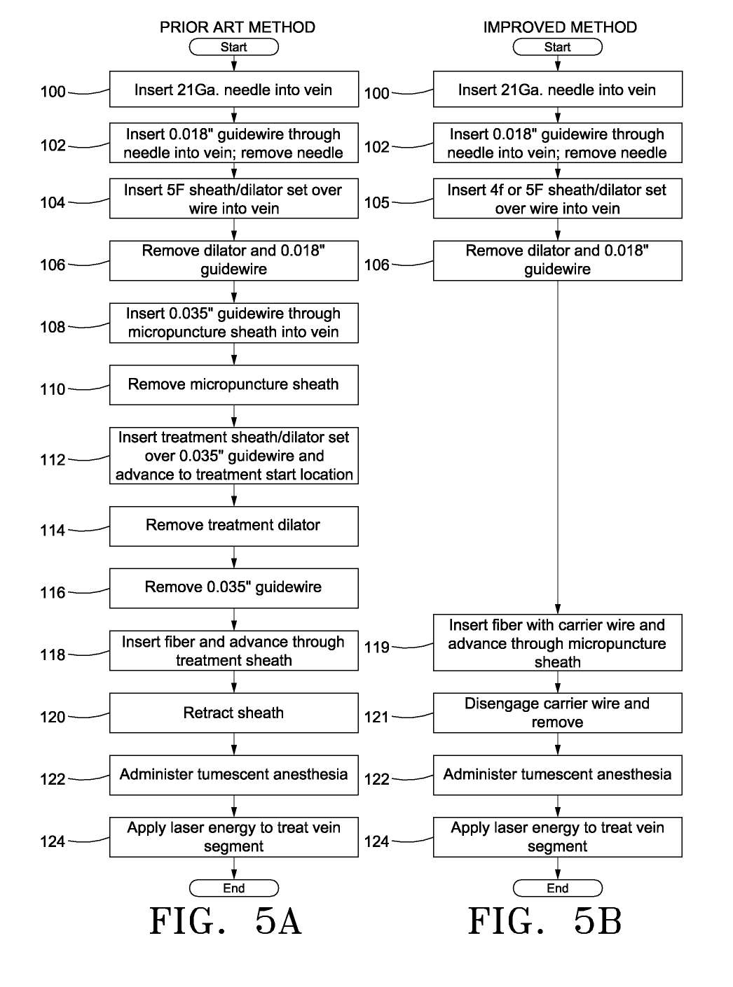

A method of using the endovascular optical fiber device 3 with carrier wire device 5 of the present invention for treating varicose veins will now be described with reference to the flowcharts in FIGS. 5A and 5B. As described in the background section, FIG. 5A illustrates the procedural steps of a prior art method of thermally treating varicose veins while FIG. 5B depicts the procedural steps associated with the present invention. Similar to the procedure in FIG. 5A, the initial vein access steps (100, 102, 105 and 106) of FIG. 5B according to the present invention are similar to steps (100, 102, 104 and 106), except the size of the sheath/dilator set inserted over the 0.018″ guidewire.

With the prior art method, a 5 F sheath/dilator assembly is typically required in order to provide sufficient dilation of the entry site to accommodate the subsequent introduction of 6 F or larger treatment sheath (see step 112). With the method of the present invention, however, a treatment sheath is not required. Accordingly, the insertion site does not require dilation larger than the diameter of a 5 F sheath. Thus, the size of the micropuncture sheath/dilator assembly may be smaller and the resulting access site puncture may be reduced relative to prior art methods. As can be appreciated by persons of ordinary skill in the art, smaller access sites are desirable as evidenced by lower rates of patient complications including hematoma, bleeding and infection.

Using the method of the present invention, starting with the distal tip 15 of the carrier wire device 5, the endovascular treatment device 1 (the optical fiber device 3 coupled to the carrier wire device 5) is inserted into the vein through the 4 F or 5 F micropuncture sheath (step 119) until the energy emitting front face 29 reaches the distal end of the target vein segment to be treated. The treatment device 1 is advanced forward through the vessel using the carrier wire tip 15 to facilitate advancement and tracking through even tortuous vessels. Because the carrier wire shaft 17 with distal tip 15, which is a spherically shaped tip, can easily track through the vessel without accessory components, numerous prior art procedure steps may be eliminated. For example, with the prior art method of use, a 0.035″ guidewire is inserted and advanced through the vessel (step 108), after which the 5 F micropuncture sheath is removed (step 110). In prior art methods, the 0.035″ guidewire is necessary in order to insert and advance the treatment sheath, which is typically a 6 F or larger size sheath (step 112). Also under the prior art method, before inserting the optical fiber, the dilator and guidewire are removed (steps 114 and 116). Consequently, a total of five steps (steps 108, 110, 112, 114 and 116) required under the prior art procedure are eliminated by the present treatment procedure.

Once the energy emitting front face 29 is positioned at the target vein area and verified by an imaging technique such as ultrasound, the carrier wire device 5 is detached from the fiber. This is done by first pulling the releasable coupler 21 in a radial direction away from the handle 9 while the handle is being held stationery. Once the coupler 21 is released from the handle 9 of the optical fiber device 3, the carrier wire shaft 17 is gently pulled in a proximal direction relative to the optical fiber device 3 to release or disengage the coupling of the wire-to-sleeve connector extension 19 from the key slot 27 of the protective sleeve 7 (step 121) and the carrier wire device 5 is withdrawn from the vessel. As the carrier wire device 5 is withdrawn through the vessel, the vein becomes irritated due to movement of the carrier wire shaft 17, wire-to-sleeve connector 11 and the carrier wire coil tip 13. The irritation in turn causes the vein to spasm and shrink.

Once the carrier wire device 5 is removed from the target vessel, tumescent anesthesia is administered along the entire vein segment being treated (step 122). Tumescent fluid is typically injected into the tissue adjacent to the vein, in an amount sufficient to provide the desired anesthetic effect, desired reduction in diameter of the vessel and the thermal insulation of the vein from heat generated by the energy emitting front face 29. According to the invention, because the carrier wire device 5 being withdrawn tends to reduce the diameter of the vessel in step 121, less tumescent fluid volume may be necessary.

Once tumescent fluid has been injected, the optical fiber device 3 is pulled back through the vein segment to be treated while energy is emitted from the emitting face 29 at the distal tip of the fiber (step 125).

With the prior art method, a treatment sheath together with the optical fiber are withdrawn with the fiber tip being slightly exposed outside the treatment sheath. As can be appreciated, even a slight misalignment of the fiber tip may result in thermal energy being transferred to the tip of the treatment sheath, resulting in potential damage to the sheath and patient complications. With the method of the present invention, however, there is no treatment sheath to be withdrawn together with the optical fiber device 5 and there is no issue of fiber tip alignment with a sheath. Accordingly, the present invention reduces potential patient complications that may be caused by a misaligned fiber tip relative to the treatment sheath.

Prior to applying laser energy in step 124, the micropuncture sheath may be removed from the vein if desired. In step 124, a laser generator (not shown) is activated, and the device is withdrawn through the vein segment, at a rate of about 1-3 millimeters per second. The laser energy produces localized thermal injury to the endothelium and vein wall causing occlusion of the vein. The laser energy travels down the optical fiber 17 through the energy-emitting face 29 of the fiber and into the vein lumen, where the laser energy is absorbed by the blood and, in turn, converted to thermal energy to substantially uniformly heat the vein wall along a 360 degree circumference, thus damaging vein wall tissue, causing cell necrosis, and ultimately causing collapse of the vessel.

In an alternative aspect, steps 121 and 122 in FIG. 5B may be reversed with the tumescent anesthesia fluid administration step being performed before the step of disengaging and removing the carrier wire device.

The process of controlling the device's pull back speed through the vessel in the case of the prior art method is typically controlled by the use of graduated markings on the treatment sheath. Since a treatment sheath is not used with the present method, the physician's pullback speed may be controlled by either markings 34 (see FIG. 2A) positioned along the fiber shaft or by using an automated pullback mechanism.

The procedure for treating the varicose vein is considered to be complete when the desired length of the target vein has been exposed to laser energy. Normally, the laser generator is turned off when the fiber tip is approximately 3 centimeters from the access site. The physician can monitor the location of the fiber tip relative to the puncture site by the presence of distinguishing marks on the distal segment of the fiber 4. Once the unique marks appear at the skin surface, the generator is turned off and the optical fiber device 3 can then be removed from the body.

The invention disclosed herein has numerous advantages over prior art treatment devices and methods. Use of the carrier wire eliminates multiple procedure steps required in prior art methods. Accessory components necessary to complete the prior art procedure steps such as the treatment sheath also are eliminated, thus enabling a reduction in overall cost of the device. Since the procedure is simplified, there may be less time required by the physician to perform the procedure. The carrier wire with its leading round tip 15 not only provides a mechanism for easily tracking and advancing the fiber through even tortuous anatomy, but also facilitates the alignment of the fiber emitting face relative to the source of reflux if desired. Another advantage of the method of the invention is that tumescent anesthesia fluid volume can be reduced since the carrier wire induces spasm and constriction of the vein prior to injection of anesthesia.

Although the device and method described herein focus on endovenous treatment using laser energy, other thermal energy forms may be used. For example, in one such alternative embodiment the energy emitting section 8 includes one or more RF coils or other electrodes for emission of RF energy. In another alternative embodiment, the energy emitting section 8 includes one or more microwave antennas located on a distal portion of the optical fiber for emitting microwave energy. As may be recognized by those of ordinary skill in the pertinent art, blood vessels other than the great saphenous vein and other hollow anatomical structures can be treated using the device and/or methods of the invention disclosed herein.

The above disclosure is intended to be illustrative and not exhaustive. This description will suggest many modifications, variations, and alternatives that may be made by those of ordinary skill in this art without departing from the scope of the invention. Those familiar with the art may recognize other equivalents to the specific embodiments described herein. Accordingly, the scope of the invention is not limited to the foregoing specification.

What is claimed is:

1. An endovenous thermal treatment device comprising:
a longitudinal energy delivery device having a distal energy emitting section;
a carrier wire having a flexible distal section with a distal tip; and
a coupler that couples the longitudinal energy delivery device and the carrier wire such that the distal tip of the flexible distal section of the carrier wire is positioned distally of the energy emitting section.

2. The device of claim 1, wherein the coupler includes:
a connector attached to the carrier wire and having an extension element ; and
a slot disposed on the longitudinal energy delivery device and adapted to receive the extension element to couple the longitudinal energy delivery device and the carrier wire.

3. The device of claim 1, wherein the coupler includes:
an extension element attached to the carrier wire and extending in a longitudinal and distal direction;
a longitudinal key slot disposed on the longitudinal energy delivery device and opening in a proximal direction; and
the extension element is adapted to be inserted into the key slot to removably connect the carrier wire to the delivery device.

4. The device of claim 1, wherein the coupler includes:
an extension element attached to one of the longitudinal energy delivery device and the carrier wire; and
a slot disposed on the other of the longitudinal energy delivery device and the carrier wire, and adapted to receive the extension element to couple the longitudinal energy delivery device and the carrier wire.

5. The device of claim 4, wherein:
the extension element extends in a longitudinal and distal direction;
the slot opens in a proximal direction; and
the extension element is adapted to be inserted into the slot to removably connect the carrier wire to the longitudinal energy delivery device.

6. The device of claim 1, further comprising a second coupler disposed proximally of the coupler and adapted to couple the longitudinal energy delivery device and the carrier wire at a location proximal of the coupler.

7. The device of claim 6, wherein the second coupler includes a lumen through which a shaft of the carrier wire is mounted.

8. The device of claim 6, wherein the second coupler includes a cylindrical recess adapted to releasably engage with the longitudinal energy delivery device.

9. The device of claim 8, wherein the longitudinal energy delivery device includes a handle adapted to releasably engage with the cylindrical recess of the second coupler.

10. The device of claim 9, wherein the handle includes a reduced diameter cylindrical portion adapted to releasably engage with the cylindrical recess of the second coupler in a tight-fitting relationship.

11. The device of claim 1, wherein the longitudinal energy delivery device includes a plurality of graduated markings positioned along the longitudinal energy delivery device.

12. The device of claim 1, wherein the flexible distal section includes a compression coil or spring.

13. The device of claim 1, wherein the distal tip includes a coil spring and a rounded portion located distally of the coil spring.

14. The device of claim 1, wherein the energy delivery device includes an optical fiber and the distal end of the optical fiber defines the energy emitting section.

15. The device of claim 1, wherein the energy emitting section includes at least one radiofrequency electrode or at least one microwave antenna.

16. An endovenous thermal treatment device comprising:
an optical fiber having a distal energy emitting section;
a carrier wire having a flexible distal section with a distal tip, and adapted to be inserted through a blood vessel; and
a coupler that couples the longitudinal energy delivery device and the carrier wire such that when the carrier wire is inserted through the blood vessel, the carrier wire carries the optical fiber.

17. An endovenous thermal treatment method comprising:
inserting into a blood vessel a carrier wire having a flexible distal section with a distal tip together with a longitudinal energy delivery device having a distal energy emitting section so as to position the energy emitting section near a treatment site of the blood vessel, the carrier wire and the longitudinal energy delivery device being coupled together when being inserted into the blood vessel with the distal tip of the flexible distal section of the carrier wire being positioned distally of the energy emitting section; and
applying thermal energy through the energy emitting section to treat the blood vessel.

18. The method of claim 17, prior to applying thermal energy, further comprising removing the carrier wire from the blood vessel while the inserted energy emitting section remains in the blood vessel.

19. The method of claim 17, wherein:
the longitudinal energy delivery device includes an optical fiber; and
the step of applying thermal energy includes applying the thermal energy through the optical fiber.

20. The method of claim 17, wherein the step of inserting includes advancing into the blood vessel the carrier wire together with the longitudinal energy delivery device without the use of a treatment sheath.

21. The method of claim 17, wherein:
the energy emitting section includes at least one radiofrequency electrode; and
the step of applying thermal energy includes applying thermal energy through the radio frequency electrode.

22. The method of claim 17, wherein:
the energy emitting section includes at least one microwave antenna; and
the step of applying thermal energy includes applying thermal energy through the microwave antenna.
